(19) United States
(12) Patent Application Publication
(10) Pub. No.: US 2013/0224884 A1
(43) Pub. Date: Aug. 29, 2013

(54) DEVICE AND METHOD FOR IMMUNOASSAYS

(71) Applicants: Helene Briand, L'Arbresle (FR); Bruno Colin, Marcy L'Etoile (FR); Cecile Paris, Bessenay (FR)

(72) Inventors: Helene Briand, L'Arbresle (FR); Bruno Colin, Marcy L'Etoile (FR); Cecile Paris, Bessenay (FR)

(73) Assignee: BIOMERIEUX, Marcy l'Etoile (FR)

(21) Appl. No.: 13/881,960

(22) PCT Filed: Nov. 16, 2011
(86) PCT No.: PCT/FR2011/052663

§ 371 Date: Apr. 26, 2013

(30) Foreign Application Priority Data
Nov. 17, 2010 (FR) 1059431

(51) Int. Cl. G01N 33/558

(52) U.S. Cl. CPC G01N 33/558
USPC 436/501; 422/69

(57) ABSTRACT

A device for performing a test to determine the presence or the absence of at least one analyte in a liquid sample includes: a support and a matrix, fixed on the support. The matrix includes: a liquid sample application zone, a marking zone and at least one reaction zone. The one reaction zone includes: a test results display zone having at least a second immobilized binding partner which is able to bind to at least one analyte, and a monitoring zone downstream of the results display zone or parallel with the results display zone which allows monitoring of the correct functioning of the device and which includes at least one analogue of the at least one analyte which is able to bind to at least a first marked binding partner. The liquid sample application zone, marking zone and reaction zone are in fluid communication.

The present invention has as its object a device and a method for performing a test known as a lateral flow test to determine the presence or the absence of an analyte in a sample. In particular, the invention relates to novel improved monitoring of the correct functioning of the device.

Lateral flow tests, also called rapid tests, are currently used in the fields of clinical, food, pharmaceutical and chemical analyses. Thus, rapid test devices are used to determine the presence of a large number of analytes, such as antibodies, antigens, hormones, proteins and chemical molecules in liquid samples. These devices generally comprise a support and a matrix which permits the migration of the liquid sample. Conventionally, a plurality of zones is distinguished in the matrix, namely an application zone of the liquid sample, a marking zone and a reaction zone, the latter comprising a capture zone and a monitoring zone. These different zones are in fluid, communication. Thus, the analyte to be detected, if it is present in the sample deposited in the application zone, binds to a first marked binding partner in the marking zone, the complex thus formed then migrates to the reaction zone where it is immobilised in the capture zone by reaction with a second binding partner and the user can determine whether the analyte is indeed present from the appearance of a detectable signal which is determined by the type of marker associated with the first binding partner. Generally, the presence of the analyte in the sample is revealed in the form of a detectable line, usually called a test line. The reaction zone also comprises a sample migration monitoring zone which will indicate to the user that at least a part of the sample has indeed passed across the matrix, upstream of the monitoring zone and in particular in the capture zone. This can be for example by the disclosure of a control line of a predetermined colour. By way of example, patent applications WO 2004/003559, WO 2006/092103, WO 2007/081330 and US 2004/0161855 can be cited. The limits of the monitoring means currently used in rapid tests on a strip, which may or may not be integrated in a cassette, are that they can only verify that the migration of the fluid has indeed taken place by capillary action from the application zone to the reaction zone and cannot monitor the correct functioning of the device and of the test.

The present invention now provides a device which integrates true positive monitoring. The positive monitoring of the invention permits verification on the one hand of the integrity and the functioning of the physical elements of the device and on the other hand verification of the functionality of the biological elements of the device, in order to monitor the correct functioning of the device and of the test.

The device of the invention comprises:
a) a support,
b) a matrix 1, fixed on the support, which allows the migration of the liquid sample, said matrix comprising:
(i) a liquid sample application zone 2,
(ii) a marking zone 3 comprising at least a first marked binding partner which is able to hind to said at least one analyte, if it is present in the liquid sample, and which is able to bind to at least one analogue of the analyte, and
(iii) at least one reaction zone 4 comprising:
a test results display zone 5 comprising at least a second immobilised binding partner which is able to bind to said at least one analyte, and
a monitoring zone 6 downstream of the results display zone 5 or parallel with the test results display zone 5 which allows monitoring of the functioning of the device and which comprises at least one analogue of the at least one analyte which is able to bind to said at least first marked binding partner;
said liquid sample application zone 2, marking zone 3 and reaction zone 4 zone being in fluid communication.

The first binding partner and the second binding partner are selected from the group consisting in antibody, mixture of antibodies, antibody fragment, mixture of antibody fragments, antibody analogue, mixture of antibody analogues, antigen, mixture of antigens, protein, mixture of proteins, polypeptide, mixture of polypeptides, peptide and mixture of peptides.

The analogue of the analyte is immobilised either directly or indirectly in the monitoring zone, or is able, in the monitoring zone, to be carried by the flow of the liquid sample to a determined region of the monitoring zone in which it is immobilised to reveal the control line.

Consequently, in one embodiment of the device of the invention, the monitoring zone 6 comprises in addition a capture reagent immobilised on the matrix of said at least one analogue to which said at least one analogue is able to bind. In particular, the capture reagent of said at least one analogue is a reagent which is identical to the second binding partner of the test results display zone.

In another embodiment of the device of the invention, in the monitoring zone 6 the analogue of the analyte is immobilised directly or indirectly on the matrix.

The analogue of the analyte can thus be immobilised on the matrix by a reagent selected from the group consisting in antibody, antibody mixture, antibody fragment, mixture of antibody fragments, antibody analogue, mixture of antibody analogues, antigen, mixture of antigens, protein, mixture of proteins, polypeptide, mixture of polypeptides, peptide, mixture of peptides, and biotin/steptavidin and biotin/avidin receptor.

The analogue of the analyte can also be immobilised on the matrix, by a capture reagent which is an identical reagent to the second binding partner.

Preferably, the analogue of the analyte is an antibody, a mixture of antibodies, an antibody fragment, a mixture of antibody fragments, an antibody analogue, a mixture of antibody analogues, an antigen, a mixture of antigens, a protein, a mixture of proteins, a polypeptide, a mixture of polypeptides, a peptide or a mixture of peptides or their associations.

Two preferred embodiments of the device according to the invention are described below:

in one embodiment the first binding partner is an antibody, an antibody fragment or an antibody analogue, the second binding partner is an antibody, an antibody fragment, or an antibody analogue and the analogue of the analyte is a protein, a polypeptide or a peptide, and

in another embodiment the first binding partner is a protein, a polypeptide or a peptide, the second binding partner is a protein, a polypeptide or a peptide, and the analogue of the analyte is an antibody, an antibody fragment or an antibody analogue.

The first binding partner is marked by a detectable marker, i.e. a compound, a substance or a particle which can be detected by visual, fluorescent, or instrumental means and in particular the detectable marker can be a coloured latex particle, a gold particle, or a magnetic particle.

The correct functioning of the device can be displayed, by the formation of a detectable positive control line which is substantially perpendicular and preferably perpendicular to the direction of the flow of the liquid sample.

In another embodiment of the device according to the invention, the latter device comprises two adjacent and parallel reaction zones 4A and 4B which are not in fluid communication with each other so that the migration of the liquid sample takes place simultaneously and independently in said zones. It is desirable, in this embodiment, for the device to comprise in addition liquid sample migration monitoring (7).

In the above-mentioned devices the monitoring zone (6) is preferably downstream of the results display zone (5).

In another embodiment of the invention, the reaction zone 4 comprises two adjacent and parallel reaction zones (4A and 4B) which are not in fluid communication with each other and the monitoring zone (6) is parallel with the results display zone (5). It is desirable in this embodiment for the device to comprise in addition monitoring of migration of the liquid sample (7).

In another particular embodiment of the device of the invention, the matrix 1 is divided into at least two adjacent and parallel parts 1A and 1B which are not in fluid communication with each other. Part 1A comprises the liquid sample application zone 2A, the marking zone 3A, the test results display zone 5, and preferably the sample migration monitoring zone 7. Part 1B comprises the liquid sample application zone 2B, the marking zone 3B and the monitoring zone 6 which permits monitoring of the device.

In a modified embodiment of the device, the matrix 1 is partially divided into two parts adjacent and parallel with each other. The matrix 1 comprises the liquid sample application zone 2 and the marking zone 3. The reaction zone 4 is divided into two adjacent and parallel parts which are not in fluid communication with each other. Zone 4A comprises the test results display zone 5, and preferably the sample migration monitoring zone 7. Zone 4E comprises the monitoring zone 6 which permits monitoring of the correct functioning of the device.

In another modified embodiment of the device of the invention, the matrix 1 is divided into at least two adjacent and parallel parts 1A and 1C which are not in fluid communication with each other. Part 1A comprises the liquid sample application zone 2, the marking zone 3A, the test results display zone 5, and preferably the liquid sample migration monitoring zone 7. Part 1B comprises an analogue application zone 9, the marking zone 3C and zone 6 which allows monitoring of the correct functioning of the device. In this embodiment, the analogue of the analyte can be present in dehydrated form in which case it is taken up by any appropriate means, for example by a buffer or by the sample. The analogue of the analyte can also be deposited in the application zone 9 in liquid form, in particular after being taken up by an appropriate liquid medium before it is deposited in the application zone 9.

### DEFINITIONS

The term “matrix” refers to any type of material which is capable of ensuring the flow and the transfer of a fluid. The transfer of the fluid can be effected by capillary force. The matrix may be, for example made of at least one bibulous material. Bibulous materials are materials which easily absorb a liquid and across which the liquid is transported by capillary action. Non-limiting examples of bibulous materials include nitrocellulose, polyester, glass fibres, etc.

“Liquid sample” means any sample taken from a patient or individual, and able to contain an analyte such as defined below. This sample can in particular be a liquid biological sample such as one of blood, serum, plasma, saliva, urine, cerebrospinal fluid, pleural fluid, or peritoneal fluid. However the biological sample also comprises semi-solid or solid samples insofar as that they can be converted into a liquid sample by any appropriate method, for example a food sample, a stool sample, a tissue sample, cell cultures, or a mucous sample. This biological sample is prepared by any type of sampling known to the man skilled in the art. The sample can also be a sample of environmental origin, i.e. a liquid, solid or semi-solid sample from the environment, such as effluents, muds, soils, plants etc . . . . Of course, when the sample is solid or semi-solid, it must be pre-treated to be converted into a liquid sample.

“Analyte” means principally an antigen, an antibody, a hormone, a protein or a chemical molecule.

When the analyte is a protein or an antigen it can be detected by binding partners, for example receptors, antibody, antibody fragments, antibody analogue and any other ligand capable of binding to a protein or to an antigen.

The binding partner antibodies are for example either polyclonal antibodies, or monoclonal antibodies.

Polyclonal antibodies can be obtained by immunisation of an animal with the appropriate immunogen, followed by the recovery of the antibodies sought in purified form, by taking serum from said animal, and separation of said antibodies from the other constituents of the serum, in particular by affinity chromatography on a column on which is fixed an antigen specifically recognised by the antibodies.

Monoclonal antibodies can be obtained by the hybridoma technique the general principle of which is below.

In a first stage, an animal, generally a mouse, is immunised with the appropriate immunogen, the B lymphocytes of which are then capable of producing antibodies against this antigen. These antibody producing lymphocytes are then fused with “immortal” myeloma cells (mouse cells in the example) to produce hybridomas. From the heterogeneous mixture of the cells thus obtained, a selection is then performed of the cells capable of producing a particular antibody and of reproducing indefinitely. Each hybridoma is reproduced in clone form, each leading to the production of a monoclonal antibody the recognition properties of which with regard to the protein will be testable for example by ELISA, by immunotransfer (Western blot) in one or two dimensions, by immunofluorescence, or using a biocaptor. The monoclonal antibodies thus selected are subsequently purified in particular by the affinity chromatography technique described above.

The monoclonal antibodies can also be recombinant antibodies obtained by genetic engineering, by techniques well known to the man skilled in the art.

“Antibody analogues” means biological and/or chemical compounds which have the same binding abilities as the antibodies or antibody fragments or similar binding capacities. In particular antibody analogues include small proteins which like antibodies are capable of binding to a biological target thus permitting its detection, its capture or quite simply its targeting in an organism or a biological sample. The fields of applications of these antibody analogues are practically as vast as those of the antibodies. By way of example can be cited the Nanofitines™, small proteins marketed by the company AFFILOGIC.

The specific binding partners of the protein or of the antigen sought in the method of the invention can be used as a capture reagent, as a detection reagent or as capture and detection reagents.

The display of the immunological reactions, i.e. of the protein/binding partner or antigen/binding partner binding can be performed by any detection means employing marking of the binding partner.

Marking means the fixing of a marker reagent capable of generating a detectable signal, i.e. a compound, a substance or a particle which can be detected by visual, fluorescent or instrumental means.
A non-limiting list of these marker reagents consists in:
   metallic or alloy particles, such as particles of colloidal gold,
   polymer particles, such as coloured latex particles,
   magnetic particles,
   fluorescent molecules,
   chemoluminescent molecules.

In the embodiments of the invention, the signal generated in the results display zone and the signal generated in the positive monitoring zone will, preferably, be of the same nature and will exhibit the same colours.

By way of an example of immunological tests such as defined above, can be cited the “sandwich” and “competitive” methods.

### FIGURES

FIG. 1:

FIG. 1A is a view from above of an embodiment of the device of the invention, before application of the sample. The device of the invention comprises a support (not shown), a matrix 1 comprising a sample application zone 2, a marking zone 3, a reaction zone 4 comprising a test results display zone 5 comprising means for displaying the test results and a positive monitoring zone 6, downstream of the test results display zone 5, comprising means for performing positive monitoring and means for displaying the correct functioning of the device and of the test. Optionally, the reaction zone 4 can in addition comprise a migration monitoring zone 7 and means for displaying the sample migration. Optionally, the matrix 1 can also comprise a sample absorption zone 8.

FIG. 1B shows the results obtained with the device of FIG. 1A after application of a negative sample for an analyte to be determined.

FIG. 1C shows the results obtained with the device of FIG. 1A after application of a positive sample for an analyte to be determined.

FIG. 1D shows a particular case in which the user cannot produce results.

Although shown respectively at 7 and 8 in FIGS. 1B to 1D, the migration monitoring zone 7 and absorption monitoring zone 8 are optional in this embodiment.

FIG. 2:

FIG. 2 is a side view of the device of FIG. 1A.

FIG. 3:

FIG. 3 is a view from above of a particular embodiment of the device of the invention, in which the matrix 1 is in the form of a U on its side. The matrix 1 comprises the necessary means to display the test results in zone 5 and means for performing the positive monitoring and for displaying the correct functioning of the device and of the test. In FIG. 3, the reaction zone 4 is shown in grey. The absorption zone 8 is optional. FIG. 3 shows the results obtained after application of a positive sample for an analyte to be determined.

FIG. 4:

FIG. 4 shows an embodiment of the invention, seen from above, in which the matrix 1 is divided into two adjacent and parallel parts 1A and 1B which are not in fluid communication with each other. The matrix 1 comprises a sample application zone 2 divided into two parts 2A and 2B, a marking zone divided into two parts 3A and 3E and a reaction zone divided into two parts 4A and 4B. Reaction zone 4A comprises the test results display zone 5, a sample migration monitoring zone 7 and the means for displaying the test results and for displaying the sample migration. Reaction zone 4B comprises the positive monitoring zone 6, the means for performing the positive monitoring and the means for displaying the correct functioning of the device and of the test in the monitoring zone 6. The test results display zone 5 and monitoring zone 6 are adjacent and parallel, with each other. Optionally, the matrix 1 comprises in addition an absorption zone 8 divided into two parts shown respectively at 8A and 8B. The liquid sample application zone 2, marking zone 3 and reaction zone 4 are in fluid communication. FIG. 4 shows the results obtained after application of a positive sample for an analyte to be determined.

FIG. 5:

FIG. 5 shows another embodiment of the device according to the invention which includes two adjacent and parallel reaction zones 4A and 4B which are not in fluid communication with each other so that the liquid sample migration occurs simultaneously and independently in said zones. The matrix 1 comprises a sample application zone 2, a marking zone 3 and a reaction zone 4 divided into 2 parts 4A and 4B, Reaction zone 4A comprises the test results display zone 5, a sample migration monitoring zone 7 and the means for displaying the test results and for displaying the sample migration. Reaction zone 4B comprises the positive monitoring zone 6 and the means for performing the positive monitoring and for displaying the correct functioning of the device and of the test in the monitoring zone 6. The test results display zone 5 and monitoring zone 6 are adjacent and parallel with each other. Optionally the matrix 1 comprises in addition an absorption zone 8 divided into two parts 8A and 8B. The liquid sample application zone 2, marking tone 3 and reaction zone 4 are in fluid communication. FIG. 5 shows the results obtained after application of a positive sample for an analyte to be determined.

FIG. 6:

FIG. 6 shows an embodiment of the invention in which the matrix 1 is divided into two adjacent and parallel parts 1A and 1C which are not in fluid communication with each other. The matrix 1 comprises a sample application zone 2, a marking zone divided into two parts 3A and 3C and a reaction zone 4 divided into two parts 4A and 4B. Reaction zone 4A comprises the test results display zone 5, a sample migration monitoring zone 7 and the means for displaying the test results and for displaying the sample migration. Reaction zone 4B comprises the monitoring zone 6 and the means necessary to perform the positive monitoring and for displaying the correct functioning of the device and of the test. The test results display zone 5 and monitoring zone 6 are adjacent and parallel with each other. The matrix 1, in its part 1C, comprises an analogue deposition zone 9. Optionally the matrix 1 comprises an absorption zone 8 divided into two parts 8A and 8B. The liquid sample application zone 2, marking zone 3 and reaction zone 4 are in fluid communication. FIG. 6 shows the results obtained after application of a positive sample for an analyte to be determined.

### EMBODIMENTS

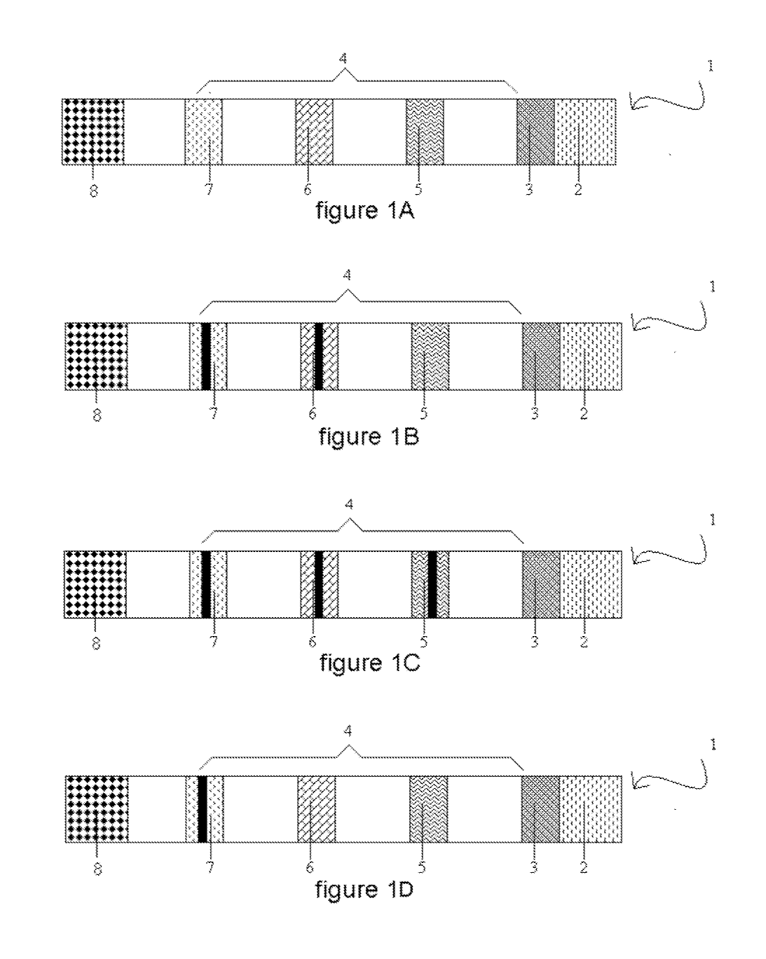

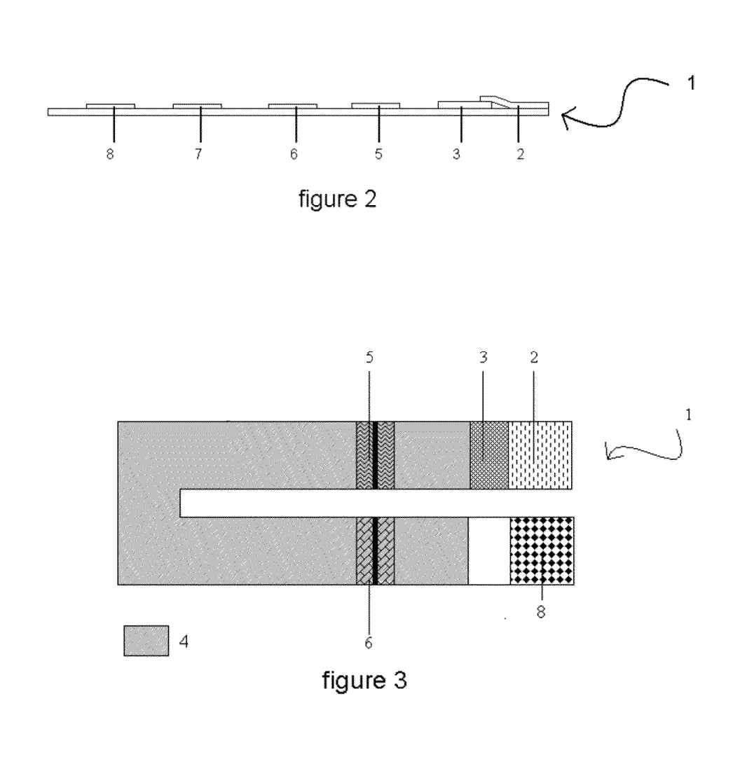

In one embodiment, with reference to FIG. 1, the matrix 1 is shown in the form of a rectangular strip the longitudinal axis of which is in the horizontal position. Zones 2, 3 and 4 are in fluid communication. Zone 3 comprises the first marked binding partner, for example an antibody carrying a visible marker, such as a particle of coloured latex, a particle of gold etc . . . . This reagent can migrate freely across the matrix in the presence of the liquid sample deposited in zone 2 and react with the analyte (antigen) to be determined if it is present. In zone 5 of the matrix 1, the second binding partner, for example an antibody having a specificity for an epitope of the antigen which is different from that recognised by the first marked antibody, is immobilised. In zone 6 of the matrix 1, an analogue of the antigen is either immobilised directly or indirectly or can migrate freely in the presence of the fluid flow in zone 6 until it is immobilised by a capture reagent which is identical to the second antibody.

FIGS. 1B and 1C show the functioning of the test in the presence of negative control and of positive samples.

As shown in FIG. 1B, the sample being a negative control, there is no emission of a detectable signal in the results display zone 5. On the contrary, there is emission of a detectable signal in the positive monitoring zone 6, revealed, for example, by a line perpendicular to the direction of the flow of the liquid sample, which means on the one hand that the negative control sample has indeed migrated to zone 6 and on the other hand the device is operational.

As shown in FIG. 1C, the sample being positive. There is emission of a detectable signal in the results display zone 5 which is revealed by a line perpendicular to the direction of flow of the liquid sample. There is also emission of a detectable signal in the positive monitoring zone 6, revealed, for example, by a line perpendicular to the direction of the flow of the liquid sample, which means on the one hand that the sample has indeed migrated and on the other hand that the device is operational. The intensity of the signal in zones 5 and 6 will be a function of the sample load, as explained in more detail in the following examples.

As shown in FIG. 1D, there is no emission of detectable signal either in the test results display zone 5, or in the monitoring zone 6. The results are uninterpretable and the test must be performed again. In the case in which migration monitoring is provided in zone 7, the display of a migration signal allows it to be established that these results are not due to physical malfunction of the device.

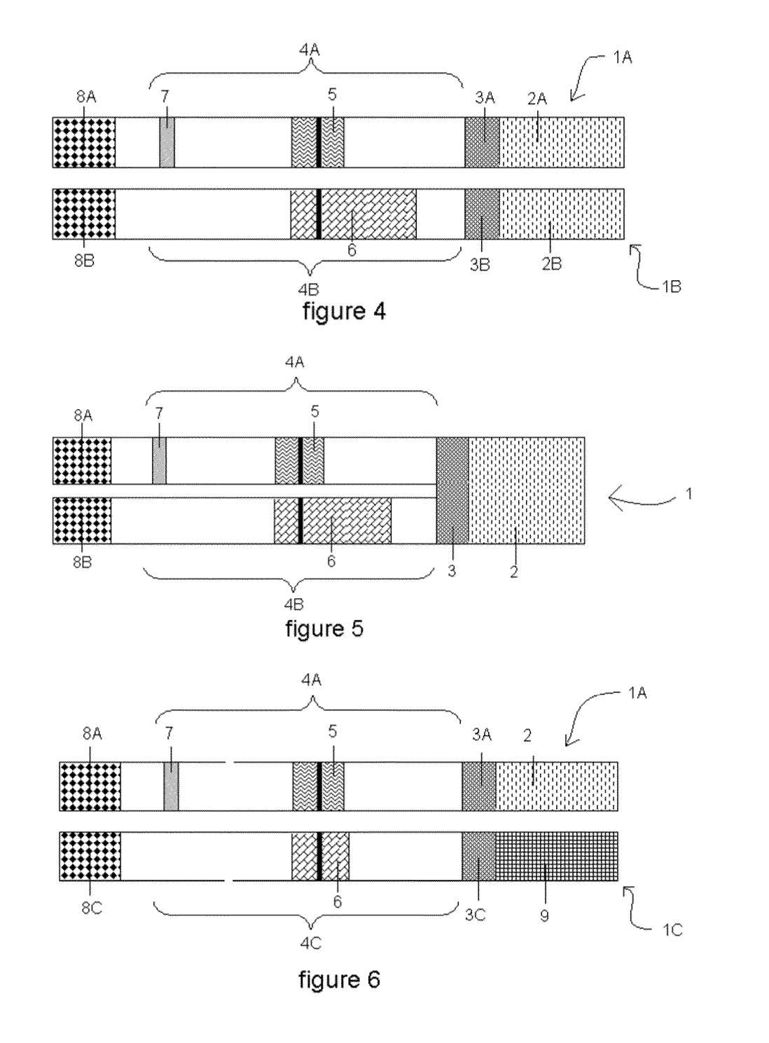

In another embodiment shown in FIG. 4, the matrix 1 is shown in the form of two strips 1A and 1B, the longitudinal axes of which are in the horizontal position. The two strips 1A and 1B are adjacent and parallel and are not in fluid communication with each other. Zones 2A, 3A and 4A are in fluid communication. Zones 2B, 3B and 48 are in fluid communication. Zones 3A and 3B respectively comprise the first marked binding partner, for example an antibody carrying a visible marker, such as a particle of coloured latex, a particle of gold etc . . . . This reagent can migrate freely across the matrix 1A in the presence of the liquid sample deposited in the application zone 2A and react with the analyte (antigen) to be determined if it is present. In zone 5 of the matrix 1A, the second binding partner, for example an antibody having a specificity for an epitope of the antigen which is different from that recognised by the first marked antibody, is immobilised. If the antigen is present, there is emission of a detectable signal in the results display zone 5 which is revealed, for example, by a line perpendicular to the direction of the flow of the liquid sample, as shown in FIG. 4. In addition, whether the sample is negative or positive, a sample migration signal must appear in the migration monitoring zone 7. The matrix 1B comprises in addition an analogue of the antigen which is either immobilised directly or indirectly or can migrate freely in the presence of the flow of the sample in zone 6 until it is immobilised by a capture reagent which is identical to the second antibody.

In another embodiment shown in FIG. 5, the liquid sample is deposited in the sample application zone 2 by any appropriate means. After application, the liquid sample starts to migrate across the matrix, enters into contact with the first marked binding partner (marked antibody) in the marking zone 3 so that a marked analyte/first binding partner complex is formed if the analyte is present in the sample. The marked complex migrates with the flow of the sample to reaction zones 4A and 4B respectively. The marked complex is on the one hand immobilised in the test display zone 5 by binding with the second specific binding partner of the analyte (antigen), which is immobilised in this zone, so that a signal is generated in the results display zone, preferably in the form of a line perpendicular to the direction of displacement of the sample. The remaining fluid, comprising the first marked binding partner and the marked complex on the other hand migrates to the monitoring zone 6 comprising an analogue of the antigen which is either immobilised directly or indirectly in the matrix, or which is free to migrate with the flow of the sample until it is immobilised in zone 6 by a capture reagent which is identical to the second antibody, so that a signal is generated in monitoring zone 6, preferably in the form of a line perpendicular to the direction of displacement of the fluid and parallel with the test line.

In another embodiment and with reference to FIG. 6, the matrix 1 is composed of two adjacent and parallel strips 1A and 1B which are not in fluid communication with each other. Zones 2A, 3A and 4A are in fluid communication. Zones 2C, 3C and 4C are in fluid communication. Zones 3A and 3B respectively comprise the first marked binding partner, for example an antibody carrying a visible marker, such as a particle of coloured latex, a particle of gold etc . . . . This reagent can migrate freely across the matrix 1A in the presence of the liquid sample deposited in the application zone 2A and react with the analyte (antigen) to be determined if it is present. In zone 5 of the matrix 1A, the second binding partner, for example an antibody having a specificity for an epitope of the antigen which is different from that recognised by the first marked antibody, is immobilised. It the antigen is present, there is emission of a detectable signal in the results display zone 5 which is revealed, for example, by a line perpendicular to the direction of the flow of the liquid sample. In addition, whether the sample is negative or positive, a sample migration signal must appear in the migration monitoring zone 7. The matrix 1C comprises the analogue of the antigen which is either deposited in liquid form in a deposition zone 9, or is present in dry form in zone 9 and is reconstituted by any appropriate means. The matrix 1C comprises in addition an analogue of the antigen which is either immobilised directly or indirectly or can migrate freely in the presence of the flow of liquid in zone 6 until it is immobilised by a capture reagent which is identical to the second antibody,

### EXAMPLES

### Example 1

### Testing for the Antigen HBs

Testing for the antigen HBs by the test illustrated in FIG. 1 consists in a sandwich type immunological reaction in one step based on an immunochromatographic technique.

Red latex particles marketed by the company Magsphère (trade name) are coated with a mixture of two anti-BBs monoclonal antibodies (bioMérieux, 2G2G10A12 and 6H6B6), at a respective concentration of 500 μg/ml. The particles are then distributed by means of a BIODOT (trade name) apparatus on a polyester membrane (Ahlstrom—trade name). The membrane is dried for one night at 37° C.

The capture antibody is a polyclonal goat anti-HBs, produced by biomérieux, which is coated on a nitrocellulose membrane CN 140 (Sartorins—trade name) at a concentration of 1 mg/ml. The distribution is performed with the BIODOT apparatus.

The positive control of the HBs Ag test or analogue of the native HBs antigen is a recombinant HBs antigen (batch 101011FF004), developed by biomérieux, which is immobilised directly, in the monitoring zone, at a concentration of 1 mg/ml by distribution with a BIODOT apparatus on the nitrocellulose membrane, at a distance of 5 mm from the polyclonal anti-HBs capture antibody. After distribution of the capture antibody and of the positive control of the test, the membrane is dried for one night at 37° C.

The two polyester and nitrocellulose membranes are then assembled on a rapid test support (backing, of the company G&L (trade name)). They are mounted in the cassettes after cutting into strip form.

The samples tested are well-characterised positive samples for the HBs Ag antigen.

Dilutions in negative serum (Scantibodies—trade name) are performed to obtain high, medium and low levels of positivity. The negative sample tested corresponds to a negative serum pool from the Etablissement Francais du Sang (EFS) of the Rhône-Alpes region.

The reading time after deposition of the sample in the sample deposition well of the cassette is 15 minutes.

Reading is performed visually by means of a reading card which is used to attribute signal intensities depending on the intensity of the red colour observed.

This card is graduated from L1 to L10. A sample is considered positive if a red colour appears with an intensity corresponding to at least L3 on the reading scale.

The results are presented in table 1 below:

TABLE 1

| | HBs Ag batch 101011FFU04 (bioMérieux) | |
|---|---|---|
| | Test Line | Positive Control Line |
| Samples | 15 minutes | 15 minutes |
| Map60 (high positive) | L7 | L7 |
| Map59 (high positive) | L7 | L7 |
| Map60 (medium positive) | L5 | L8 |
| Map59 (medium positive) | L5 | L8 |
| Map64 (low positive) | L4 | L8 |
| Map60 (low positive) | L4 | L8 |
| Negative serum pool | L1 | L8 |

The results show that in the case of a negative sample, only the positive control is detected with a high colour intensity (L8). This result provides confirmation that the absence of signal at the test line 5, which corresponds to the capture polyclonal, is due to the negativity of the sample and not to a functional defect of the cassette used for the testing. Indeed, in case of a negative serum, the monoclonal antibodies coupled to the red particles are available and a complex is formed with the recombinant HBs antigen upon migration of the particles at the control line 6.

Conversely, in case of a positive sample, the particles fixing the antigen present in the sample form a complex with the capture polyclonal at the test line 5, depending on the level of positivity of the sample, and antibodies at the particle can remain available and form a second complex at the control line 6.

### Example 2

### Testing for the Influenza A Antigen

Testing for the influenza A antigen is based on the same principle as that described in example 1 for testing for the HBs Ag antigen. An anti-influenza A monoclonal detection antibody (bioMérieux, 15C9H2) is immobilised on red particles (Magsphère). The same antibody is used for capture on the nitrocellulose membrane. The positive control (analogue of the antigen) is a recombinant protein (Nucleoprotein Influenza A batch 101011FFU05, bioMérieux) immobilised directly on the nitrocellulose membrane in the monitoring zone, at a concentration of 1 mg/ml. The analogue is distributed by the BIODOT apparatus on the nitrocellulose membrane at a distance of 5 mm from the anti-influenza A capture monoclonal. After assembly and mounting in the cassettes, the tests are performed with a reading time at 10 minutes after deposition of the sample.

A concentration range of the recombinant protein (Nucleoprotein INF A, bioMérieux) was tested. The negative sample tested is a PBS buffer.

The results are presented in table 2 below:

TABLE 2

| | Influenza A batch 101011FFU05 (bioMérieux) | |
|---|---|---|
| | Test Line | Positive Control Line |
| Samples | 10 minutes | 10 minutes |
| NP A INF (40 μg/ml) | L10 | L6 |
| NP A INF (1 μg/ml) | L10 | L8 |
| NP A INF (50 ng/ml) | L7 | L10 |
| NP A INF (10 ng/ml) | L4 | L10 |
| bioMérieux buffer | L1 | L10 |
| batch 100923FFU03 | | |

The results show that in the case of a negative sample, only the positive control is detected with a high colour intensity (L10). This result provides confirmation that the absence of signal at the test line 5 (corresponding to the capture monoclonal), is due to the negativity of the sample and not to a functional defect of the cassette used for the testing. Indeed, in case of a negative serum, the monoclonal antibodies coupled to the red particles are available and a complex is formed with the recombinant Influenza A protein upon migration of the particles at the control line 6. Conversely, in case of a positive sample, the particles fixing the antigen present in the sample form a complex with the capture monoclonal at the test line 5. Depending on the level of positivity of the sample tested, (from 40 μg/ml to 10 ng/ml) , antibodies on the particle can remain available and form a second complex at the control line 6.

### Example 3

### Testing for Anti-HIV-1 Group M Antibody

The detection is based on the same principles as those described in examples 1 and 2, i.e. one-step sandwich type immunochromatographic testing. The only difference resides in the fact that in this test the presence of an antibody is tested for, i.e. an anti-HIV-1 group M antibody.

Blue latex particles marketed by the company VARIAN (trade name) are coated with specific peptides of the HIV-1 group M virus. These particles are then distributed on a polyester membrane (Ahlstrom). The capture peptides are coated on the nitrocellulose membrane (Millipore, 135UF).

The positive control of the HIV test is an anti-HIV-1 group M monoclonal antibody (bioMérieux, P12G11B10) coated on the nitrocellulose membrane at a concentration of 1 mg/ml. The positive control antibody is distributed by the apparatus at a distance of 5 mm from the capture peptides.

After distribution of the capture peptides and of the positive control of the test, the membrane is dried for one night at 37° C.

After assembly and mounting in the cassettes, the tests are performed. The reading of the signal is taken 30 minutes after the deposition of the sample.

The samples tested are well-characterised HIV positive samples. The negative sample is a negative serum pool from EFS of the Rhône Alpe region.

The results are presented in table 3 below:

TABLE 3

| | HIV ½ batch 101011FFU03 | |
|---|---|---|
| | | Positive |
| | Test Line | Control Line |
| | Reading time | Reading time |
| HIV ½ | 30 minutes | 30 minutes |
| MARHIV0023 (SCI13) HIV1 M | L10 | L1 |
| MARHIV0024 (SCI14) HIV1 M | L10 | L1 |
| MARHIV0025 (SCI15) HIV2 | L9 | L6 |
| MARHIV0032 (SCI22) HIV group O | L8 | L6 |
| Negative serum pool | L1 | L6 |

The results show that in the case of a negative sample, only the positive control is detected. This result provides confirmation that the absence of signal at the test line 5 (corresponding to the capture peptides), is due to the negativity of the sample and not to a functional defect of the cassette used for the testing. Indeed, in case of a negative serum, the HIV peptides coupled to the blue particles are available and a complex is formed with the control anti-HIV monoclonal antibody upon migration of the particles at the control line 6.

Conversely, in case of a positive sample, the particles fixing the anti-HIV antibodies present in the sample form a complex, with the capture peptides at the test line 5, depending on the level of positivity of the sample, the peptides on the particle are saturated or remain partially available and form a second complex at the control line 6.

1. Device for performing a test to determine the presence or the absence of at least one
analyte in a liquid sample comprising:
a) a support,
b) a matrix fixed on the support, which allows the migration of the liquid sample, said matrix comprising:
(i) a liquid sample application zone.
(ii) a marking zone comprising at least a first marked binding partner which is able to bind to said at least one analyte, if it is present in the liquid sample, and which is able to bind to at least one analogue of the analyte, and
(iii) at least one reaction zone comprising:
a test results display zone comprising at least a second immobilised binding partner which is able to bind to said at least one analyte, and
a monitoring zone downstream of the results display zone or parallel with the results display zone which allows monitoring of the correct functioning the device and which comprises at least one analogue of said at least one analyte which is able to bind to said at least first marked binding partner;
said liquid sample application zone, marking zone and reaction zone being in fluid communication.

2. Device according to claim 1, in which the first binding partner and the second binding partner are selected from the group consisting in antibody, antibody mixture, antibody fragment, mixture of antibody fragments, antibody analogue, mixture of analogues, antigen, mixture of antigens, protein, mixture of proteins, polypeptide, mixture of polypeptides, peptide and mixture of peptides.

3. Device according to claim 1, in which the monitoring zone comprises in addition a capture reagent of the analogue, immobilised on the matrix, and to which said one analogue is able to bind.

4. Device according to claim 1, in which in the zone the analogue is immobilised on the matrix directly or indirectly.

5. Device according to claim 3, in which the capture reagent of the analogue is a reagent which is identical to the second binding partner of the test results display zone.

6. Device according to claim 4, in which the analogue of the analyte is immobilised on the matrix by a reagent selected from the group consisting in antibody, mixture(s) of antibodies, antibody fragment, mixture of antibody fragments, antibody analogue, mixture of antibody analogues, antigen, mixture of antigens, protein, mixture of proteins, polypeptide and mixture of polypeptides, peptide, mixture of peptides, and biotin/steptavidin and biotin/avidin receptor.

7. Device according to claim 1, in which the analogue of the analyte is selected from the group consisting in antibody, mixture of antibodies, antibody fragment, mixture of antibody fragments, antibody analogue, mixture of antibody analogues, antigen, mixture of antigens, protein, mixture of proteins, polypeptide, mixture of polypeptides, peptide and mixture of peptides.

8. Device according to claim 1, in which
(i) the first binding partner is an antibody, an antibody fragment or an antibody analogue, the second binding partner is an antibody, an antibody fragment or an antibody analogue and the analogue of the analyte is a protein, a polypeptide or a peptide, or
(ii) the first binding partner is a protein, a polypeptide or a peptide, the second binding partner is a protein, a polypeptide or a peptide, and the analogue of the analyte is an antibody, an antibody fragment or an antibody analogue.

9. Device according to claim 8, in which the analogue of the analyte can be immobilised on the matrix, by a capture reagent which is an identical reagent to the second binding partner.

10. Device according to claim 1, in which the first binding partner is marked by a detectable marker.

11. Device according to claim 10, in which the detectable marker is a compound, a substance or a particle which can be detected by visual, fluorescent or instrumental means and in particular the detectable marker can be a coloured latex particle, a gold particle, or a magnetic particle.

12. Device according to claim 1 in which the correct functioning of the device and of the test is revealed in the form of a detectable line which is substantially perpendicular to the direction of the flow of the liquid sample.

13. Device according to claim 1, in which the monitoring zone is downstream of the results display zone.

14. Device according to claim 1, in which the reaction zone 4 comprises two adjacent and parallel reaction zones which are not in fluid communication with each other.

15. Device according to claim 14, in which the monitoring zone is parallel with the results display zone.

16. Device according to claim 14, comprising in addition monitoring of liquid sample migration monitoring.

17. Method for performing a test to determine the presence or the absence of an analyte in a liquid sample comprising the steps of:
placing the liquid sample in contact with a device such as defined in claim 1 and determining whether the sample does not comprise or comprises the analyte by reference to the absence or to the presence of a detectable signal in the test results display zone, and when there is no signal in the test results display zone, confirming the correct functioning of the test by reference to the presence of a signal in the monitoring zone.
